(19) United States
(12) Patent Application Publication
(10) Pub. No.: US 2005/0011016 A1
(43) Pub. Date: Jan. 20, 2005

(54) Means for colouring keratin fibres comprising 2-amino-5-ethylphenol

(76) Inventors: Cecile Pasquier, Marly (CH); Veronique Buclin, Morlon (CH); Hans-Juergen Braun, Ueberstorf (CH); Guido Sauter, Thoerishaus (CH)

Correspondence Address: Striker Striker & Stenby, 103 East Neck Road, Huntington, NY, 11743, US

(21) Appl. No.: 10/497,872

(22) PCT Filed: Oct. 17, 2003
(86) PCT No.: PCT/EP03/11526

(30) Foreign Application Priority Data
Nov. 7, 2002 (DE) 102 51 830.0

(51) Int. Cl. A61K 7/13

(52) U.S. Cl. 8/405

(57) ABSTRACT

The present patent application has for an object colorants for dyeing keratin fibers, particularly human hair, and containing 2-amino-5-ethylphenol or a physiologically compatible, water-soluble salt thereof

The present invention relates to colorants for oxidative dyeing of keratin fibers, particularly human hair, containing 2-amino-5-ethylphenol as a dye component.

In the area of keratin fiber dyeing, particularly hair dyeing, oxidation dyes have attained substantial importance. In this case, the coloration is produced by reaction of certain developers with certain couplers in the presence of an appropriate oxidant. Suitable developers are, in particular, 2,5-diaminotoluene, 2,5-diaminophenylethyl alcohol, p-aminophenol, 1,4-diaminobenzene and 4,5-diamino-1-(2-hydroxyethyl)pyrazole, and suitable couplers are, for example, resorcinol, 2-methyl-resorcinol, 1-naphthol, 3-aminophenol, m-phenylenediamine, 2-amino-4-(2′-hydroxyethyl)aminoanisole, 1,3-diamino-4-(2′-hydroxyethoxy)benzene and 2,4-diamino-5-fluorotoluene.

The oxidation dyes used for dyeing human hair must meet numerous requirements in addition to producing hair colorations that are stable for at least 4 to 6 weeks. For example, these dyes must be harmless from a toxicological and dermatological standpoint, and the hair colorations obtained must have good light fastness, resistance to permanent waving, rubbing fastness and resistance to shampooing as well as sufficient resistance to perspiration. Moreover, by combining appropriate developers and couplers, it must be possible to create a wide range of different color shades.

The adjustment of lighter color shades presents a special problem in terms of uniform dye uptake from the hair roots to the hair tips and in terms of resistance of the colorations to permanent wave treatment. The use of direct yellow-dyeing aromatic nitro dyes together with oxidative hair dye precursors represents a partial solution of the said problem, but the stability of the colorations over a period of several weeks is often unsatisfactory.

To solve the said problem, DE 28 33 989 A1 proposes the use of 2-amino-5-methylphenol as a yellow-dyeing oxidative dye in oxidative hair colorants. Although this compound is well suited as shade-adjustment agent for the creation of bright blond shades and gold shades, it does not fully meet requirements, particularly as regards the resistance of the hair colorations to the action of chemicals, for example permanent wave agents.

We have now found that by use of 2-amino-5-ethylphenol-containing oxidative colorants, the requirements placed on such agents, particularly as regards wash fastness and resistance to the action of chemicals, for example permanent wave agents, are met to an unusually high degree.

The object of the present invention therefore is a colorant for oxidative dyeing of keratin fibers, for example wool, furs, feathers or hair and particularly human hair, said colorant being characterized in that it contains 2-amino-5-ethylphenol or a physiologically compatible, water-soluble salt thereof

2-Amino-5-ethylphenol can be used as the free base as well as in the form of a physiologically compatible salt of an inorganic or organic acid, for example hydrochloric acid, sulfuric acid, phosphoric acid, acetic acid, propionic acid, lactic acid or citric acid.

The colorants of the invention contain 2-amino-5-ethylphenol in a total amount from about 0.001 to 5 weight percent, an amount from about 0.001 to 2 weight percent and particularly from 0.01 to 1 weight percent being preferred.

2-Amino-5-ethylphenol dyes keratinic material to yellow shades without the addition of other dyes.

To achieve additional color shades, common oxidative dyes, for example developers or couplers alone or in admixture with one another, can be added.

Suitable couplers are, in particular, N-(3-dimethylaminophenyl)urea, 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxyethoxy)-5-methylbenzene, 2,4-di[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 1,3-diamino-4-(2,3-dihydroxypropoxy)benzene, 1,3-diamino-4-(3-hydroxypropoxy)benzene, 1,3-diamino-4-(2-methoxyethoxy)benzene, 2,4-diamino-1,5-di(2-hydroxyethoxy)benzene, 1-(2-aminoethoxy)-2,4-diaminobenzene, 2-amino-1-(2-hydroxyethoxy)-4-methylaminobenzene, 2,4-diaminophenoxyacetic acid, 3-[di(2-hydroxyethyl)amino]aniline, 4-amino-2-di[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di(2,4-diaminophenoxy)propane, di(2,4-diaminophenoxy)methane, 1,3-diamino-2,4-dimethoxybenzene, 2,6-bis-(2-hydroxyethyl)aminotoluene, 4-hydroxyindole, 3-dimethylaminophenol, 3-diethylaminophenol, 5-amino-2-methylphenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methylphenol, 3-aminophenol, 2-[(3-hydroxyphenyl)amino]-acetamide, 5-[(2-hydroxyethyl)amino]-4-methoxy-2-methylphenol, 5-[(2-hydroxyethyl)amino]-2-methylphenol, 3-[(2-hydroxyethyl)amino]phenol, 3-[(2-methoxyethyl)amino]phenol, 5-amino-2-ethylphenol, 5-amino-2-methoxyphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydroxypyridine, 2,6-dihydroxy-3,4-dimethylpyridine, 5-amino-4-chloro-2-methylphenol, 1-naphthol, 2-methyl-1-naphthol, 1,5-dihydroxynaphthalene, 1,7-dihydroxynaphthalene, 2,3-dihydroxynaphthalene, 2,7-dihydroxynaphthalene, 2-methyl-1-naphthol acetate, 1,3-dihydroxybenzene, 1-chloro-2,4-dihydroxybenzene, 2-chloro-1,3-dihydroxybenzene, 1,2-dichloro-3,5-dihydroxy-4-methylbenzene, 1,5-dichloro-2,4-dihydroxybenzene, 1,3-dihydroxy-2-methylbenzene, 3,4-methylenedioxyphenol, 3,4-methylenedioxyaniline, 5-[(2-hydroxyethyl)amino]-1,3-benzodioxole, 6-bromo-1-hydroxy-3,4-methylenedioxybenzene, 3,4-diaminobenzoic acid, 3,4-dihydro-6-hydroxy-1,4(2H)-benzoxazine, 6-amino-3,4-dihydro-1,4(2H)benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole and 2,3-indolinedione.

Preferred developers are 1,4-diaminobenzene (p-phenylenediamine), 1,4-diamino-2-methylbenzene (p-toluylenediamine), 1,4-diamino-2,6-dimethylbenzene, 1,4-diamino-3,5-diethylbenzene, 1,4-diamino-2,5-dimethylbenzene, 1,4-diamino-2,3-dimethylbenzene, 2-chloro-1,4-diaminobenzene, 1,4-diamino-2-(thiophen-2-yl)benzene, 1,4-diamino-2-(thiophen-3-yl)benzene, 4-(2,5-diaminophenyl)-2-[(diethyl-amino)methyl]thiophene, 2-chloro-3-(2,5-diaminophenyl)thiophene, 1,4-diamino-2-(pyridin-3-yl)benzene, 2,5-diaminobiphenyl, 2,5-diamino-4′-(1-methylethyl)-1,1′-biphenyl, 2,3′,5-triamino-1,1′-biphenyl, 1,4-diamino-2-methoxymethylbenzene, 1,4-diamino-2-aminomethylbenzene, 1,4-diamino-2-[(phenylamino)methyl]benzene, 1,4-diamino-2-{[ethyl-(2-hydroxyethyl)amino]methyl}benzene, 1,4-diamino-2-hydroxymethylbenzene, 1,4-diamino-2-(2-hydroxyethoxy)benzene, 2-[2-(acetylamino)ethoxy]-1,4-diaminobenzene, 4-phenylaminoaniline, 4-dimethylaminoaniline, 4-diethylaminoaniline, 4-dipropylaminoaniline, 4-[ethyl-(2-hydroxyethyl)amino]aniline, 4-[di(2-hydroxyethyl)amino]aniline, 4-[di(2-hydroxyethyl)amino]-2-methylaniline, 4-[(2-methoxyethyl)amino]aniline, 4-[(3-hydroxypropyl)-amino]aniline, 4-[(2,3-dihydroxypropyl)amino]aniline, 4-{[(4-aminophenyl)methyl]amino}aniline, 4-[(4-aminophenylamino)methyl]phenol, 1,4-diamino-N-(4-pyrrolidin-1-ylbenzyl)benzene, 1,4-diamino-N-furan-3-ylmethylbenzene, 1,4-diamino-N-thiophen-2-ylmethylbenzene, 1,4-diamino-N-furan-2-yl-methylbenzene, 1,4-diamino-N-thiophen-3-ylmethylbenzene, 1,4-diamino-N-benzylbenzene, 1,4-diamino-2-(1-hydroxyethyl)benzene, 1,4-diamino-2-(2-hydroxyethyl)benzene, 1,4-diamino-2-(1-methylethyl)benzene, 1,3-bis[(4-aminophenyl)(2-hydroxyethyl)amino]-2-propanol, 1,4-bis[(4-aminophenyl)-amino]butane, 1,8-bis(2,5-diaminophenoxy)-3,6-dioxaoctane, 2,5-diamino-4′-hydroxy-1,1′-biphenyl, 2,5-diamino-2′-trifluoromethyl-1,1′-biphenyl, 2,4′,5-triamino-1,1′-biphenyl, 4-aminophenol, 4-amino-3-methylphenol, 4-amino-3-(hydroxymethyl)phenol, 4-amino-3-fluorophenol, 4-methylaminophenol, 4-amino-2-(aminomethyl)phenol, 4-amino-2-(hydroxymethyl)phenol, 4-amino-2-fluorophenol, 4-amino-2-[(2-hydroxyethyl)amino]methylphenol, 4-amino-2-methylphenol, 4-amino-2-(methoxymethyl)phenol, 4-amino-2-(2-hydroxyethyl)phenol, 5-aminosalicylic acid, 2,5-diaminopyridine, 2,4,5,6-tetra-aminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 4,5-diamino-1-pentyl-1H-pyrazole, 4,5-diamino-1-(phenylmethyl)-1H-pyrazole, 4,5-diamino-1-[(4-methoxyphenyl]methyl-1H-pyrazole, 2-aminophenol, 2-amino-6-methylphenol, 2-amino-5-methylphenol, 1,2,4-trihydroxybenzene, 2,4-diaminophenol, 1,4-dihydroxybenzene and 2-{[(4-aminophenyl)amino]methyl)}-1,4-diaminobenzene.

The aforesaid developers and couplers can be used in the colorants of the invention either alone or in admixture with each other, the total amount of developers and couplers in the colorant of the invention being about 0.01 to 12 weight percent and particularly about 0.2 to 6 weight percent.

Moreover, the colorant of the invention can contain other dye components, for example 4-(2,5-diaminobenzylamino)aniline or 3-(2,5-diaminobenzylamino)aniline, as well as common natural dyes, dyes identical to natural ones, or synthetic direct dyes from the group consisting of anionic (acid) and cationic (basic) dyes, triarylmethane dyes, nitro dyes, disperse dyes and azo dyes, for example natural dyes such as indigo or henna, triphenylmethane dyes such as 4-[(4′-aminophenyl)-(4′-imino-2″,5″-cyclohexadien-1″-ylidene)methyl]-2-methylaminobenzene monohydrochloride (C.I. 42 510), and 4-[(4′-amino-3′-methylphenyl)-(4″imino-3″-methyl-2″,5″-cyclohexadien-1″-ylidene)methyl]-2-methylaminobenzene monohydrochloride (C.I. 42 520), aromatic nitro dyes such as 4-(2′-hydroxyethyl)aminonitrotoluene, 2-amino-4,6-dinitrophenol, 2-amino-5-(2′-hydroxyethyl)aminonitrobenzene, 2-chloro-6-(ethylamino)-4-nitrophenol, 4-chloro-N-(2-hydroxyethyl)-2-nitroaniline, 5-chloro-2-hydroxy-4-nitroaniline, 2-amino-4-chloro-6-nitrophenol and 1-[(2′-ureidoethyl]amino-4-nitrobenzene, azo dyes such as sodium 6-[(4′-aminophenyl)azo]-5-hydroxynaphthalene-1-sulfonate (C.I. 14 805) and disperse dyes, for example 1,5-diaminoanthraquinone and 1,4,5,8-tetraaminoanthraquinone.

The colorant can contain the aforesaid other dye components in a total amount of about 0.1 to 4 weight percent.

The aforesaid developers and/or couplers and/or other dye components in combination with the 2-amino-5-ethylphenol of the invention make it possible to obtain many different color shades. For example, blond to brown hair colorations can be achieved by use of a combination of the 2-amino-5-ethylphenol of the invention and 4-(2,5-diaminobenzylamino)aniline and/or 2-(3-aminophenyl)-aminomethyl-1,4-diaminobenzene or the salts thereof

Naturally, the additional couplers, developers and other dye components, provided they are bases, can also be used in the form of their physiologically compatible salts of organic or inorganic acids, for example hydrochloric acid or sulfuric acid, or—if they contain aromatic OH— groups—in the form of salts of bases, for example as alkali metal phenoxides.

Moreover, if the colorants are used for dyeing hair, they can also contain common cosmetic additives, for example antioxidants such as ascorbic acid, thioglycolic acid or sodium sulfite, as well as perfume oils, complexing agents, wetting agents, emulsifiers, thickeners and hair-care agents.

The colorant of the invention can be formulated, for example, as a solution, particularly an aqueous or aqueous-alcoholic solution, or as a paste, cream, gel, emulsion or aerosol preparation. Such a colorant preparation consists of a mixture of dye components and additives commonly used for such preparations.

Common additives to solutions, creams, emulsions or gels are, for example, solvents such as water, lower aliphatic alcohols, for example ethanol, propanol or isopropanol, glycerol or glycols such as 1,2-propylene glycol; moreover wetting agents or emulsifiers from the classes of anionic, cationic, amphoteric or nonionic surface-active substances such as, for example, the fatty alcohol sulfates, ethoxylated fatty alcohol sulfates, alkylsulfonates, alkylbenzenesulfonates, alkyltrimethyl-ammonium salts, alkylbetaines, ethoxylated fatty alcohols, ethoxylated nonylphenols, fatty alkanolamides and ethoxylated fatty esters, furthermore thickeners such as the higher fatty alcohols, starch, cellulose derivatives, petrolatum, paraffin oil and fatty acids; moreover hair-care agents such as cationic resins, lanolin derivatives, cholesterol, pantothenic acid and betaine. The. said constituents are used in amounts commonly employed for such purposes, for example the wetting agents and emulsifiers at a concentration from about 0.5 to 30 weight percent, the thickeners in an amount from about 0. 1 to 30 weight percent and the hair-care agents at a concentration from about 0.1 to 5 weight percent.

Depending on the composition, the colorant of the invention can be weakly acidic, neutral or alkaline. In particular, it has a pH from 6.5 to 11.5, the adjustment to a basic value preferably being achieved with ammonia or an organic amine, for example monoethanolamine or triethanolamine, or an amino acid, or an inorganic base such as sodium hydroxide or potassium hydroxide. It is also possible to use combinations of the aforesaid compounds, particularly a combination of ammonia and monoethanolamine. For pH adjustment in the acidic range, an inorganic or organic acid, for example phosphoric acid, acetic acid, citric acid or tartaric acid, can be used.

For use in oxidative dyeing of hair, the afore-described colorant is mixed with an oxidant just before use and the resulting mixture is applied to the hair in an amount sufficient for the hair treatment, in general from about 60 to 200 grams, depending on the fullness of the hair.

Suitable oxidants for developing the hair coloration are mainly hydrogen peroxide or the compounds of addition thereof to urea, melamine, sodium borate or sodium carbonate in the form of a 3 to 12%, preferably 6% aqueous solution. Atmospheric oxygen can also be used. If a 6% hydrogen peroxide solution is used as the oxidant, the weight ratio of hair colorant to oxidant is from 5:1 to 1:2 and preferably 1:1. Larger amounts of oxidant are used primarily at higher dye concentrations in the hair colorant or when more pronounced hair bleaching is wanted at the same time. The mixture is allowed to act on the hair at 15 to 50° C. for about 10 to 45 minutes, preferably for 30 minutes, after which the hair is rinsed with water and dried. Optionally, following this rinsing the hair is washed with a shampoo and optionally post-rinsed with a weak organic acid, for example citric acid or tartaric acid. The hair is then dried.

The colorant of the invention containing 2-amino-5-ethylphenol gives colorations of excellent color stability, particularly in terms of light fastness, wash fastness, rubbing fastness and resistance to permanent waving. As far as the coloring properties are concerned, depending on the kind and composition of the dye components, the colorant of the invention provides a wide range of different color shades ranging from blond to brown, purple, violet, blue and black. Said shades are characterized by high color intensity and good color balancing between damaged and undamaged hair. Furthermore, the very good coloring properties of the hair colorant of the present patent application are, in particular, characterized by the fact that this colorant also makes it possible to dye gray, chemically not previously damaged hair with good covering power and without any problems.

2-Amino-5-ethylphenol can be prepared by known methods by nitration of 3-ethylphenol or a 3-ethylphenol derivative followed by reduction of the nitro group to the amino group. Such a method of preparation is described, for example, in WO 96/25157 A1 (Example 92 a/b).

The following examples are intended to illustrate the subject matter of the invention more closely without limiting its scope.

### EXAMPLES

### Examples 1 to 15

### Hair Colorants

Hair colorant solutions of the following composition were prepared:

| | | |
|---|---|---|
| X | g | of 2-amino-5-ethylphenol |
| U | g | of developer E8 to E15 as per Table 1 |
| Y | g | of coupler K11 to K35 as per Table 3 |
| Z | g | of direct dye D1 to D3 as per Table 2 |
| 10.0 | g | of potassium oleate (8% aqueous solution) |
| 10.0 | g | of ammonia (22% aqueous solution) |
| 10.0 | g | of ethanol |
| 0.3 | g | of ascorbic acid |
| to 100.0 | g | water |

Just before use, 30 g of the above colorant solution was mixed with 30 g of a 6% aqueous hydrogen peroxide solution. The mixture was then applied to bleached hair. After an exposure time of 30 minutes at 40° C., the hair was rinsed with water, washed with a commercial shampoo and dried. Table 5 summarizes the coloring results.

### Examples 16 to 21

### Hair Colorants

Dye carriers in cream form and having the following composition were prepared:

| | | |
|---|---|---|
| X | g | of 2-amino-5-ethylphenol |
| U | g | of developer E8 to E15 as per Table 1 |
| Y | g | of coupler K11 to K35 as per Table 3 |
| Z | g | of direct dye D1 to D3 as per Table 2 |
| 15.0 | g | of cetyl alcohol |
| 0.3 | g | of ascorbic acid |
| 3.5 | g | of sodium lauryl alcohol diethylene glycol ether |
| | | sulfate, 28% aqueous solution |
| 3.0 | g | of ammonia, 22% aqueous solution |
| 0.3 | g | of sodium sulfite, anhydrous |
| to 100 | g | water |

Just before use, 30 g of the above hair colorant cream was mixed with 30 g of a 6% aqueous hydrogen peroxide solution. The mixture was then applied to hair. After an exposure time of 30 minutes at 40° C., the hair was rinsed with water, washed with a commercial shampoo and dried. Table 6 summarizes the coloring results.

### Examples 22 to 29

### Hair Colorants

Hair colorant solutions of the following composition were prepared

| | | |
|---|---|---|
| X | g | of 2-amino-5-ethylphenol |
| Z | g | of dye component W1 or W2 as per Table 4 |
| U | g | of developer E8 to E15 as per Table 1 |
| 10.0 | g | of potassium oleate (8% aqueous solution) |
| 10.0 | g | of ammonia (22% aqueous solution) |
| 10.0 | g | of ethanol |
| 0.3 | g | of ascorbic acid |
| to 100.0 | g | water |

Just before use, 30 g of the above colorant solution was mixed with 30 g of a 6% aqueous hydrogen peroxide solution. The mixture was then applied to bleached hair. After an exposure time of 30 minutes at 40° C., the hair was rinsed with water, washed with a commercial shampoo and dried. The following Table 7 summarizes the coloring results.

TABLE 1
Developers

| | |
|---|---|
| E8 | 1,4-diaminobenzene |
| E9 | 2,5-diaminophenylethanol sulfate |
| E10 | 3-methyl-4-aminophenol |
| E11 | 4-amino-2-aminomethylphenol dihydrochloride |
| E12 | 4-aminophenol |
| E13 | N,N-bis-(2′-hydroxyethyl)-p-phenylenediamine sulfate |
| E14 | 4,5-diamino-1-(2′-hydroxyethyl)pyrazole sulfate |
| E15 | 2,5-diaminotoluene sulfate |

TABLE 2
Direct Dyes

| | |
|---|---|
| D1 | 2,6-diamino-3-[(pyridin-3-yl)azo]pyridine |
| D2 | 6-chloro-2-ethylamino-4-nitrophenol |
| D3 | 2-amino-6-chloro-4-nitrophenol |

TABLE 3
Couplers

| | |
|---|---|
| K11 | 1,3-diaminobenzene |
| K12 | 2-amino-4-(2′-hydroxyethyl)aminoanisole sulfate |
| K13 | 1,3-diamino-4-(2′-hydroxyethoxy)benzene sulfate |
| K14 | 2,4-diamino-5-fluorotoluene sulfate |
| K15 | 3-amino-2-methylamino-6-methoxypyridine |
| K16 | 3,5-diamino-2,6-dimethoxypyridine dihydrochloride |
| K17 | 2,4-diamino-5-ethoxytoluene sulfate |
| K18 | N-(3-dimethylamino)phenylurea |
| K19 | 1,3-bis-(2,4-diaminophenoxy)propane tetrahydrochloride |
| K21 | 3-aminophenol |
| K22 | 5-amino-2-methylphenol |
| K23 | 3-amino-2-chloro-6-methylphenol |
| K24 | 5-amino-4-fluoro-2-methylphenol sulfate |
| K25 | 1-naphthol |
| K26 | 1-acetoxy-2-methylnaphthalene |
| K31 | 1,3-dihydroxybenzene |
| K32 | 2-methyl-1,3-dihydroxybenzene |
| K33 | 1-chloro-2,4-dihydroxybenzene |
| K34 | 4-(2′-hydroxyethyl)amino-1,2-methylenedioxybenzene. |
| | HCl |
| K35 | 3,4-methylenedioxyphenol |

TABLE 4
Dye Components

| | |
|---|---|
| W1 | 4-(2,5-diaminobenzylamino)aniline.HCl |
| W2 | 2-(3-aminophenyl)aminomethyl-1,4-diaminobenzene.HCl |

TABLE 5
Hair Colorants

| | Example No. | | | | |
|---|---|---|---|---|---|
| | 1 | 2 | 3 | 4 | 5 |
| Dyes | (Dye quantity in grams) | | | | |
| 2-Amino-5- | 0.30 | 0.03 | 0.05 | 0.03 | 0.02 |
| ethylphenol | | | | | |
| E10 | | | | 0.55 | |
| E11 | | 0.55 | | | |
| E12 | | | 0.55 | | |
| E14 | | | | | 0.55 |
| K31 | | | | 0.18 | 0.20 |
| K32 | | 0.22 | | | |
| K33 | | | 0.20 | | |
| K25 | | 0.30 | | 0.30 | 0.30 |
| K26 | | | 0.35 | | |
| Coloring result | bright | red- | red- | red- | red-brown |
| | yellow | brown | brown | brown | |

Example No.

| | |
|---|---|
| 6 | 7 |

| | |
|---|---|
| | (Dye quantity |
| Dyes | in grams) |

| | | |
|---|---|---|
| 2-Amino-5-ethylphenol | 0.01 | 0.005 |
| E8 | 0.10 | 0.10 |
| E9 | 0.25 | |
| E15 | | 0.25 |
| K13 | 0.09 | 0.09 |
| K31 | 0.20 | |
| K32 | | 0.20 |
| K21 | 0.05 | |
| K22 | | 0.05 |
| Coloring result | blond | blond |

| | Example | | | | | |
|---|---|---|---|---|---|---|
| | 8 | 9 | 10 | 11 | 12 | 13 |
| Dyes | (Dye quantity in grams) | | | | | |
| 2-Amino-5- | 0.010 | 0.006 | 0.020 | 0.005 | 0.050 | 0.010 |
| ethylphenol | | | | | | |
| E9 | | | | | 0.096 | 1.800 |
| E10 | 0.096 | 0.240 | 0.300 | 0.900 | 0.010 | 0.700 |
| K12 | | | | | 0.010 | |
| K18 | | | | | | 0.030 |
| K21 | | | | | 0.020 | 0.060 |
| K22 | 0.080 | 0.200 | 0.250 | 0.056 | | 0.580 |
| K25 | | | | | 0.030 | |
| K31 | | | | 0.200 | | 0.800 |
| K32 | | 0.030 | 0.050 | 0.316 | | |
| K35 | 0.018 | | | | | |
| D2 | | | | 0.010 | | |
| D3 | 0.040 | 0.060 | 0.025 | | | |
| Coloring | light | copper- | bright | purple- | silver- | dark- |
| result | blond to | gold | copper | brown | blond | mahogany |
| | copper | | | | | |
| | gold | | | | | |

Example

| | |
|---|---|
| 14 | 15 |

| | |
|---|---|
| | (Dye quantity in |
| Dyes | grams) |

| | | |
|---|---|---|
| 2-Amino-5-ethylphenol | 0.03 | 0.15 |
| E8 | | 0.50 |
| E10 | 0.60 | 0.05 |
| K12 | | 1.10 |
| K17 | | 1.10 |
| K22 | 0.50 | |
| K23 | | 0.60 |
| K32 | 0.03 | |
| D1 | 0.25 | |
| D2 | | 0.50 |
| D3 | 0.15 | |
| Coloring result | orange | red-orange |

In the tables reproduced from the original document, commas indicate decimal points - Translator

TABLE 6
Hair Colorants

Example No.

| | | |
|---|---|---|
| 16 | 17 | 18 |

| | |
|---|---|
| Dyes | (Dye quantity in grams) |

| | | | |
|---|---|---|---|
| 2-Amino-5-ethylphenol | 0.10 | 0.05 | 0.01 |
| E8 | 0.70 | 0.70 | 0.70 |
| K12 | 0.10 | 0.10 | 0.10 |
| K13 | | | |
| K23 | 0.10 | 0.10 | 0.10 |
| K31 | 0.40 | 0.40 | 0.40 |
| D2 | 0.10 | 0.10 | 0.10 |
| Coloring result | brown | brown | brown |

Example No.

| | | |
|---|---|---|
| 19 | 20 | 21 |

| | |
|---|---|
| Dyes | (Dye quantity in grams) |

| | | | |
|---|---|---|---|
| 2-Amino-5-ethyl- | 0.005 | 0.270 | 0.010 |
| phenol | | | |
| E8 | 0.250 | | |
| E9 | | 1.710 | 0.020 |
| E10 | 2.000 | 0.200 | 0.010 |
| K13 | | 0.100 | |
| K16 | | | 0.015 |
| K21 | | 0.800 | |
| K22 | 1.800 | | 0.250 |
| K23 | | 0.200 | |
| K26 | | | 0.030 |
| K31 | 0.250 | 0.135 | 0.020 |
| D2 | | 0.010 | |
| Coloring result | orange | chocolate- | silver- |
| | | brown | blond |

TABLE 7

| | Example No. | | | | | | | |
|---|---|---|---|---|---|---|---|---|
| | 22 | 23 | 24 | 25 | 26 | 27 | 28 | 29 |
| Dyes | (Dye quantity in grams) | | | | | | | |
| 2-Amino-5- | 0.01 | 0.18 | 0.04 | 0.18 | 0.18 | 0.18 | 0.06 | 0.18 |
| ethylphenol | | | | | | | | |
| E8 | | 0.12 | | 0.12 | | | | |
| E9 | | | 0.12 | | 0.15 | | | |
| E15 | | | | | | 0.13 | | |
| W1 | 0.90 | | | 0.38 | | 0.38 | 0.38 | 0.38 |
| W2 | | 0.37 | 0.05 | | 0.58 | | | |
| Coloring | deep- | medium. | medium. | black | brown | black | medium | brown |
| result | blue | brown | blond | brown | | brown | brown | |

1. Colorant for oxidative dyeing of keratin fibers, characterized in that it contains 2-amino-5-ethylphenol or a physiologically compatible, water-soluble salt thereof.

2. Colorant according to claim 1, characterized in that it contains 2-amino-5-ethylphenol in an amount from 0.001 to 5 weight percent.

3. Colorant according to claim 1 characterized in that additionally it contains at least one dye from the group consisting of developers, couplers, direct dyes and other dye components.

4. Colorant according to claim 3, characterized in that the additional dye is selected from among 4-(2,5-diaminobenzylamino)aniline and 2-(3-aminophenyl)aminomethyl-1,4-diaminobenzene and the salts thereof.

5. Colorant according to claim 1, characterized in that it has a pH from 6.5 to 11.5.

6. Method for oxidative dyeing of hair, characterized in that before use a colorant according to claim 1 is mixed with an oxidant, applied to the hair and allowed to act at a temperature from 15 to 50° C. for 10 to 45 minutes, and the hair is then rinsed with water, optionally washed with a shampoo and then dried.
